(19) United States
(12) Patent Application Publication
(10) Pub. No.: US 2015/0150973 A1
(43) Pub. Date: Jun. 4, 2015

(54) MAGNESIUM PHOSPHATE GELS

(71) Applicant: THE ROYAL INSTITUTION FOR THE ADVANCEMENT OF LEARNING/MCGILL UNIVERSITY, MONTREAL (CA)

(72) Inventors: Jake Barralet, Montreal (CA); Faleh Tammi, Montreal (CA)

(21) Appl. No.: 14/388,463

(22) PCT Filed: Mar. 28, 2013
(86) PCT No.: PCT/CA2013/050250

§ 371 Date: Sep. 26, 2014

Related U.S. Application Data
(60) Provisional application No. 61/618,059, filed on Mar. 30, 2012.

(51) Int. Cl. A61K 47/02; A61K 31/196; A61K 9/70

(52) U.S. Cl. CPC A61K 47/02; A61K 9/7007; A61K 31/196

(57) ABSTRACT

There is provided a magnesium phosphate gel comprising water as a dispersing phase and phosphate ions (PO₄³⁻), a divalent cation, and sodium ions (Na⁺), wherein the divalent cation is magnesium (Mg²⁺) or a mixture of magnesium and calcium (Ca²⁺), the mixture comprising up to 30% by weight of calcium based on the total weight of the mixture. Methods of making and manufacturing this gel are also provided.

### CROSS REFERENCE TO RELATED APPLICATIONS

This application claims benefit, under 35 U.S.C. §119(e), of U.S. provisional application Ser. No. 61/618,059, filed on Mar. 30, 2012.

### FIELD OF THE INVENTION

The present invention relates to magnesium phosphate gels. More specifically, the present invention is concerned with magnesium phosphate gels, dehydrated magnesium phosphate gels and xerogels produced from these gels.

### BACKGROUND OF THE INVENTION

In the search for alternative drug delivery methods to enhance compliance and improve safety, researchers have discovered that the permeability of mucous membranes provides a convenient route for the systemic delivery of new and existing drugs. Transmucosal delivery offers the potential for once daily dosage, avoids the effects of first pass metabolism, and can provide as much as four times the absorption rate of drugs delivered transdermally. The improved bioavailability allows more accurate and lower dosing and fewer side effects. There are a number of transmucosal formulation platforms currently in existence. Most focus on one specific type of administration such as BEMA polymer film (BioDelivery Sciences International), RapidMist™ aerosol (Generex Biotechnology), OraDisc™ film (Uluru), OraVescent™ tablet (Cima Labs), Transmucosal Film (Auxilium Pharmaceuticals) for oral drug delivery, ChiSys™ (West Pharmaceutical Services) and Intravail™ (Aegis Therapeutics) for nasal delivery. These formulations are generally washed out or they dissolve after a certain length of exposure. Transmucosal delivery has been mainly used to administer hormones such as insulin, calcitonin and estrogens or nitroglycerin, and opiates such as fentanyl or morphine for pain management.

On the other hand, topical mucosal delivery is a route of choice to administer a drug destined to treat a mucosa.

In adhesive pharmaceutical drugs, such as muco-adhesive drugs, topical mucosal or transmucosal drug delivery agents should ideally be biocompatible, bioadhesive, thixotropic and bioresorbable.

Most mucoadhesive polymers, such as carbopol and hydroxypropyl methyl cellulose, do not possess both thixotropy and bioresorption properties. This limits their usefulness as drug delivery additives. Currently, very few synthetic mucoadhesive-thixotropic polymers are FDA approved as drug additives. These include hydroxy-ethylcellulose (HEC), polycarbophil (PC), poly(vinylpyrrolidone) (PVP), poloxamer 407 (P407), carbopol 934P (C934P), and propolis extract (PE). None of these polymers is resorbable in vivo. On the other hand, resorbable mucoadhesive polymers such as chitosan lack thixotropic properties. Currently, there are no FDA approved materials combining all the above properties.

Water-based gels (hydrogels) have a wide range of biomedical applications such as drug delivery, food additives and cell therapy. While numerous organic hydrogels have been developed, only a limited number of inorganic systems exhibit hydrogel-like properties; well-known examples being silica gel, aluminum based gels and the V₂O₅— based hydro- and aerogels. Most inorganic hydrogels cannot be used for biomedical applications due to toxicity, impurities, extreme pH levels, instability under physiological conditions, and/or their lack of bioresorption.

In particular, the use of silicate based thixotropic clays (such as Laponite Clay) in the food and drug industry is indeed very limited due to their lack of resorption in the body. These materials are rather used to improve the performance and properties of a wide range of industrial and consumer products. More specifically, layered silicates are used as film formers and rheology modifiers. They are thus added to waterborne products, such as surface coatings, household cleaners and personal care products, to impart thixotropic properties, shear sensitive viscosity and improved stability and syneresis control.

### SUMMARY OF THE INVENTION

In accordance with the invention, there is provided:
1. A magnesium phosphate gel comprising water as a dispersing phase and phosphate ions (PO₄³⁻), a divalent cation, and sodium ions (Na⁺), wherein the divalent cation is magnesium (Mg²⁺) or a mixture of magnesium and calcium (Ca²⁺), the mixture comprising up to 30% by weight of calcium based on the total weight of the mixture.
2. The magnesium phosphate gel of item 1, comprising the phosphate ions, the divalent cation and sodium ions at mole fractions of about 0.33 to about 0.44, about 0.03 to about 0.09, and about 0.48 to about 0.63, respectively.
3. The magnesium phosphate gel of item 2, comprising the phosphate ions, the divalent cation and sodium ions at mole fractions of about 0.36 to about 0.42, about 0.05 to about 0.08, and about 0.50 to about 0.57, respectively.
4. The gel of any one of items 1 to 3, comprising more than about 50% by weight of water as the dispersing phase based on the total weight of the gel.
5. The gel of item 4, comprising more than about 70% by weight of water as the dispersing phase based on the total weight of the gel.
6. The gel of item 5, comprising more than about 90% by weight of water as the dispersing phase based on the total weight of the gel.
7. The gel of item 6, comprising between about 92% and about 98% by weight of water as the dispersing phase based on the total weight of the gel.
8. The gel of item 7, comprising between about 92% and about 96% by weight of water as the dispersing phase based on the total weight of the gel.
9. The gel of any one of items 1 to 8, wherein the phosphate, magnesium, optional calcium, and sodium ions form nanosheets.
10. The gel of item 9, wherein the nanosheets are about 200 nm wide, about 10 nm thick and up to about 1 μm long.
11. The gel of item 9 or 10, wherein the nanosheets contain magnesium phosphate.
12. The gel of item 11, wherein the nanosheets contain magnesium bi- and tri-phosphate.
13. The gel of any one of items 9 to 12, wherein the nanosheets further contain hydration water.
14. The gel of item 13, wherein the nanosheets contain from about 10 to about 20% by weight of hydration water based on the water of the dried gel.
15. The gel of any one of items 1 to 14, wherein the divalent cation is the mixture of magnesium and calcium.
16. The gel of item 15, wherein the mixture comprise between about 10% and about 30% by weight of the calcium based on the total weight of the mixture.
17. The gel of any one of items 1 to 14, wherein the divalent cation is magnesium.
18. The gel of item 17, comprising phosphate, magnesium, and sodium ions at mole fractions of about 0.39, about 0.08, and about 0.53, respectively
19. The gel of item 17 or 18, further comprising up to about 200% by weight of pyrophosphate (P₂O₇)⁴⁻, based on the weight of the phosphate.
20. The gel of item 19, comprising between about 10% and about 20% by weight of pyrophosphate based on the weight of the phosphate.
21. The gel of any one of items 1 to 20, further comprising on or more of corn oil, sodium metaphosphate, sodium pyrophosphate, sodium citrate, xantham gum, sodium alginate, a carboxylate salt, a carboxylate acid, or chitosan.
22. The gel of any one of items 1 to 21, further having loaded therein a bioactive substance.
23. The gel of any one of items 1 to 22, being non-toxic.
24. The gel of any one of items 1 to 23, being thixotropic.
25. The gel of item 24, having a liquefaction stress of about 50 Pa or less.
26. The gel of item 25, having a liquefaction stress between about 30 and about 40 Pa.
27. The gel of any one of items 24 to 26, having a recovery time of 10 seconds or less.
28. The gel of item 27, having a recovery time of about 6 seconds.
29. The gel of any one of items 1 to 28, being bioadhesive.
30. The gel of any one of items 1 to 29, being bioresorbable.
31. A dehydrated or partially dehydrated magnesium phosphate gel comprising the gel of any one of items 1 to 30, wherein at least part of the water in the dispersing phase is replaced by an organic liquid once the gel is formed.
32. The dehydrated or partially dehydrated gel of item 31, wherein all of the water in the dispersing phase is replaced by the organic liquid.
33. The dehydrated or partially dehydrated gel of item 31 or 32, wherein the organic liquid is ethanol or glycerol.
34. The gel of any one of items 1 to 33 being dried so as to form a xerogel.
35. The gel of item 34, being in the form of a membrane.
36. The gel of item 35, wherein the membrane is translucent.
37. A method of manufacturing the gel of any one of items 1 to 36, the method comprising:
   (a) providing a first aqueous solution comprising sodium hydroxide,
   (b) providing a second aqueous solution comprising phosphoric acid or monomagnesium phosphate,
   (c) dissolving in the second solution, magnesium hydroxide or trimagnesium phosphate, and optionally calcium chloride or calcium hydroxide, thereby producing a third solution,
   (d) mixing together the second and third solutions, thereby producing the gel, wherein the second and third solutions provide the gel with phosphate ions (PO₄³⁻), a divalent cation, and sodium ions (Na⁺) at mole fractions of about 0.25 to about 0.375, about 0.125 to about 0.5, and about 0.25 to about 0.5, respectively, wherein the divalent cation is magnesium (Mg²⁺) or a mixture of magnesium and calcium (Ca²⁺), the mixture comprising up to 30% by weight of calcium based on the total weight of the mixture.
38. The method of item 37, wherein the mixing at step (d) occurs within 10 minutes of the dissolution at step (c).
39. The method of item 37 or 38, wherein the second solution comprises phosphoric acid.
40. The method of any one of items 37 to 39, wherein at step (c), magnesium hydroxide only is dissolved in the second solution.
41. The method of any one of items 37 to 40, being carried out at room temperature.
42. The method of any one of items 37 to 40, further comprising filtering and washing the gel.
43. The method of any one of items 37 to 42, further comprising replacing at least part of the water in the dispersing phase by an organic liquid once the gel is formed.
44. The method of item 43, wherein all of the water in the dispersing phase is replaced by the organic liquid.
45. The method of item 43 or 44, wherein the organic liquid is ethanol or glycerol.
46. The method of any one of items 37 to 45, further comprising loading a bioactive molecule in the gel.
47. The method of any one of items 37 to 46, further comprising drying the gel to form a xerogel.
48. The method of item 47, wherein the drying in carried out at room temperature.
49. Use of a gel according to any one of items 1 to 36 as a bioactive substance delivery agent.
50. The use of item 49, wherein the bioactive substance is an antibiotic.
51. The use of item 49 or 50, wherein the delivery agent is for the treatment of a periodontal disease.
52. The use of item 51, wherein the delivery agent is for the treatment of peri-implantitis.
53. The use of any one of items 49 to 52, wherein the delivery agent is an adhesive delivery agent.
54. The use of item 53, wherein the adhesive delivery agent is a mucosa adhesive delivery agent.
55. The use of item 54, wherein the delivery agent is for transmucosal delivery of the bioactive substance.
56. The use of any one of items 49 to 54, wherein the delivery agent is for topical delivery of the bioactive molecule.
57. The use of item 49 or 50, wherein the delivery agent is an injectable delivery agent.
58. The use of item 49 or 50, wherein the delivery agent is intended for oral administration.
59. The use of any one of items 49 to 58, where the bioactive substance is a drug.
60. The use of item 59, wherein the bioactive substance is an antiobiotic.
61. Use of a gel according to any one of items 1 to 36 in wound care.
62. Use of a gel according to any one of items 1 to 36 in a drug-eluting medical device.
63. Use of a gel according to any one of items 1 to 36 in a coating.
64. Use of a gel according to any one of items 1 to 36 as a coating.
65. A method of delivering a bioactive substance, the method comprising formulating the bioactive substance and a gel according to any one of items 1 to 36 into a dosage form, and administering the dosage form.
66. The method of item 65, wherein the dosage form is an adhesive dosage form.
67. The method of item 66, wherein the adhesive dosage form is a mucosa adhesive dosage form.
68. The method of item 67, wherein the dosage form delivers the bioactive substance via transmucosal absorption.
69. The method of any one of items 65 to 67 wherein the dosage form delivers the bioactive substance via topical absorption.
70. The method of item 65 wherein the dosage form is an injectable dosage form.
71. The method of item 65, wherein the dosage form is an oral dosage form.
72. The method of any one of items 65 to 71, where the bioactive substance is a drug.
73. A method of promoting healing of a wound, the method comprising applying a gel according to any one of items 1 to 36 to the wound.
74. A drug-eluting medical device comprising a gel according to any one of items 1 to 36.
75. A coating comprising a gel according to any one of items 1 to 36.

### BRIEF DESCRIPTION OF THE DRAWINGS

In the appended drawings:

FIG. 1 shows the pH before and during gel formation for three gel formulations;

FIG. 2 shows the X-ray diffractogram of the crystal product of 0.75 0.15 0.2 (top) and Newberyite (bottom);

FIG. 3 shows the X-ray diffractogram of the crystal product of 0.5 0.1 0.8 (top) and Bobierrite (bottom);

FIG. 4 shows the X-ray diffractogram of the crystal product of 0.25 0.05 0.4 (top) and Bobierrite and magnesium phosphate hydrate (bottom);

FIG. 5 shows the X-ray diffractogram of gel 0.5 0.1 1;

FIG. 6 shows the X-ray diffractogram of gel 1 0.2 1;

FIG. 7 (A) is a phase diagram presenting the nature of the precipitates obtained from sodium/phosphate/magnesium solutions; (B) is an interpolation diagram showing the pH of the different solutions as a function of the various components concentration; (C) is a photographic images of the gel (1) and the crystalline precipitate (2) obtained from the solutions presented in (A); and (D) is a phase diagram of summarizing the XRD findings of the precipitates obtained from the different solutions;

FIG. 8 is a picture showing (A) the pipetting of the gel into a beaker of distilled water, (B) a cohesive sphere formed by a droplet of the gel pipetted drop wise in water, and (C) seven gel pellets on the bottom a 20 ml beaker filled with distilled water;

FIG. 9 is an X-ray diffraction pattern of a gel heated at 700° C.;

FIG. 10 shows the TGA and DSC analysis of a washed-dried gel sample;

FIG. 11 shows the TGA and DSC analysis of an un-washed-dried gel sample;

FIG. 12 shows the infrared spectra of the hydrated gel (top) and the unwashed (middle) and washed (bottom) dried gel samples;

FIG. 13 shows photographs where a gel is injected through an insulin needle (A) and then recovering (B);

FIG. 14 shows the rheological analysis of a gel;

FIG. 15 shows crio-TEM images at (A) ×30000, (B) ×49000, (C) ×18500 and (D) ×30000 showing the gel ultrastructure;

FIG. 16 is a photograph showing a gel adhered to gastric mucosa after 24 hours of incubation in aqueous oscillating medium;

FIG. 17 is a micrograph showing live-dead assay of human bone marrow cells cultured in a gel;

FIG. 18 (A) to (C) are photographs showing xerogels (in (C) the gel is on top of McGill University coat of arms);

FIG. 19 is a graph of the release profile of diclofenac from fresh and dried gel as a function of time; and

FIG. 20 shows the application of a gel according to an embodiment of the invention in a periodontal pocket (or peri-implant pocket) to treat peri-implantitis.

### DETAILED DESCRIPTION OF THE INVENTION

### Magnesium Phosphate Gel

In accordance with the present invention, there is provided a magnesium phosphate gel. This gel comprises water as its dispersing phase. Further, this gel comprises phosphate (PO₄³⁻) ions; a divalent cation (i.e. magnesium (Mg²⁺) ions optionally with some calcium (Ca²⁺) ions); and sodium (Na⁺) ions.

The phosphate ions are typically provided by a solution of phosphoric acid (H₃PO₄) or monomagnesium phosphate (Mg(H₂PO₄)₂) in water used to make the gel. The magnesium ions are typically provided by magnesium hydroxide (Mg(OH)₂—a solid) or trimagnesium phosphate (Mg₃(PO₄)₂—another solid) that is added to the abovementioned solution. The calcium is typically provided by calcium hydroxide or calcium chloride that is also added to that solution. The sodium ions are typically provided by a solution of sodium hydroxide (NaOH) that is mixed with the magnesium-containing solution.

More specifically, the gel comprises phosphate, the divalent cation, and sodium at mole fractions of about 0.33 to about 0.44, about 0.03 to about 0.09, and about 0.48 to about 0.63, respectively. For example, the gel can comprise phosphate, the divalent cation, and sodium at mole fractions of about 0.36 to about 0.42, about 0.05 to about 0.08, and about 0.50 to about 0.57, respectively. Further examples of such gels include gels comprising the phosphate ions, the divalent cation and sodium ions at mole fractions of:

0.36, 0.07, and 0.57, respectively,

0.39, 0.08, and 0.53, respectively,

0.41, 0.05, and 0.54, respectively, and

0.42, 0.08, and 0.50, respectively.

It will be readily apparent to the skilled person that, as the above amounts of phosphate, divalent cation and sodium are given as mole fractions, the sum of these three mole fractions should be 1 (give or take the rounding errors). This is indeed the standard definition of mole fraction in the art: “In chemistry, the mole fraction is defined as the amount of a constituent divided by the total amount of all constituents in a mixture. The sum of all the mole fractions is equal to 1”. Herein, the mole fractions take only the divalent cation, phosphate and sodium into account. Water and optional additives that can be added to the gel are not considered.

In embodiments, the divalent cation is magnesium (Mg²⁺) only. In other embodiments, it is a mixture of magnesium and calcium, the mixture comprising up to 30% by weight of calcium based on the total weight of the mixture. In embodiments, the mixture comprises about 10% to about 30% by weight of calcium based on the total weight of the mixture.

In embodiments, the gel comprises phosphate, magnesium, and sodium ions at mole fractions of about 0.39, about 0.08, and about 0.53, respectively (that corresponds to the gel identified as 0.75 0.15 1 in the Examples below).

The amount of water (as a dispersing phase) in the gel is typically about 50% or more, for example 70% or more by weight based on the total weight of the gel. In embodiments, the gel may comprise more than about 90% of water, for example between about 92 and 98% or between about 92 and 96% of water as the dispersing phase.

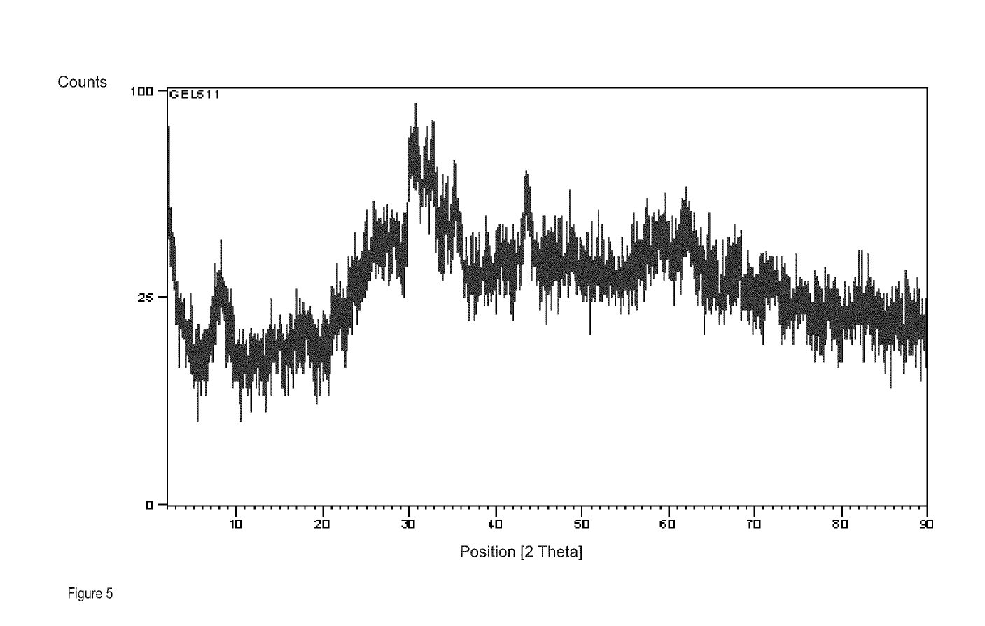

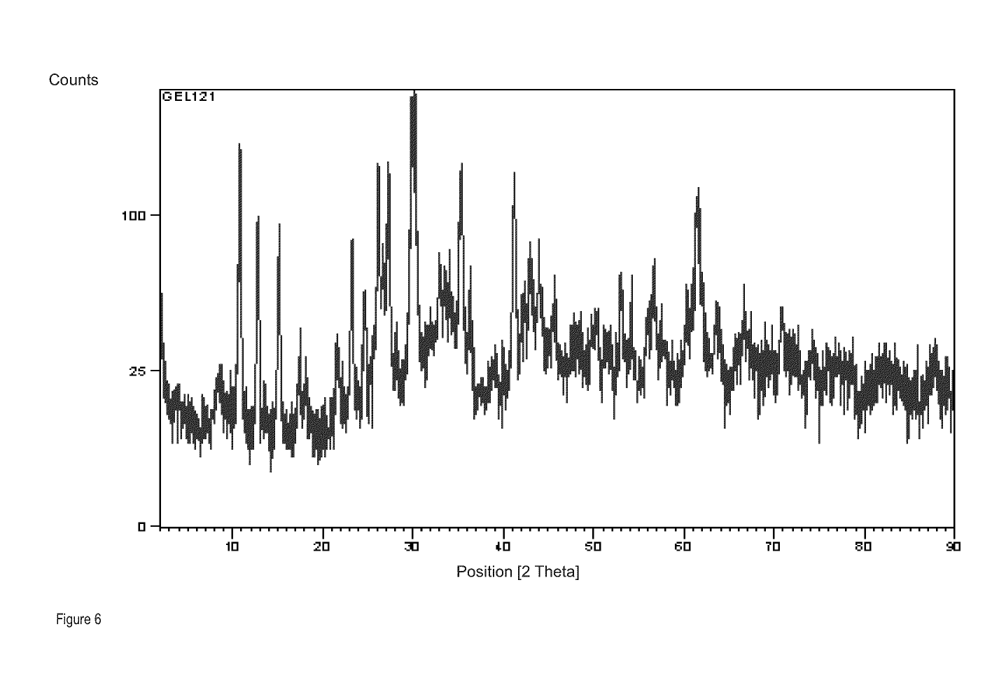

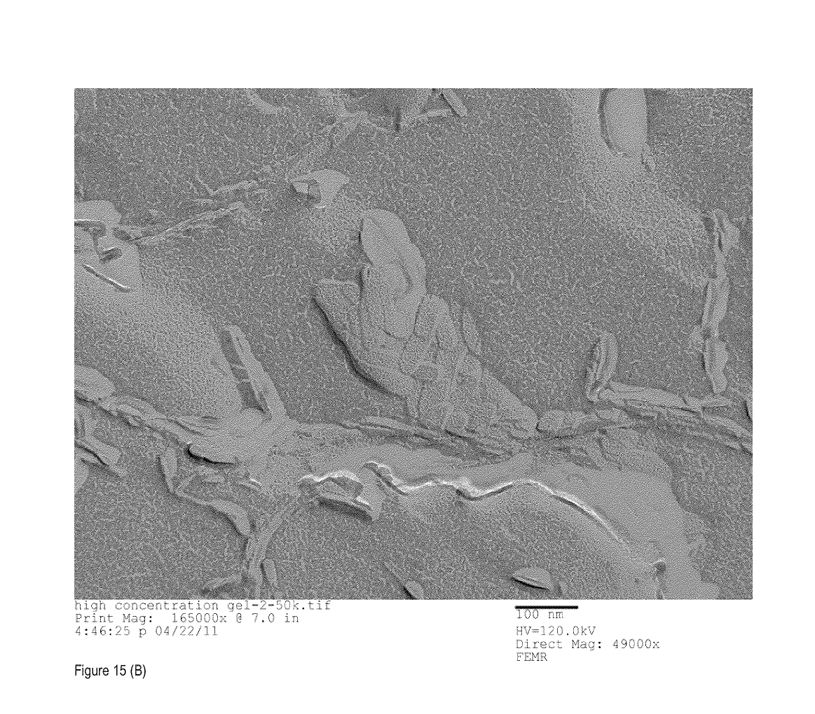

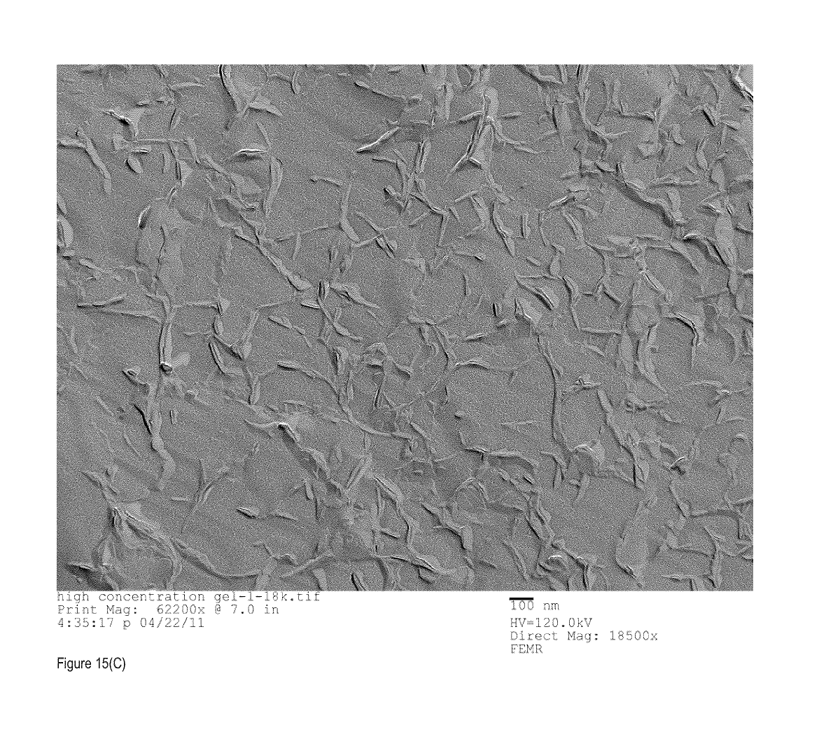

When observed by transmission electron microscopy (TEM), in embodiments, the gel appears to comprise thin nano-plates or nanosheets. More specifically, these nanosheets can be about 200 nm wide, very thin (e.g. about 10 nm thick) and up to 1 μm long. As seen by TEM, these nanosheets agglomerate, and form interconnected planes (see FIG. 15). Without being bound by theory, these nanosheets are believed to be crystalline (because of their appearance and of their X-ray diffracted pattern when dried). However, as discussed in the Examples, the hydrated gels of the invention appear to be amorphous when analyzed by X-ray diffraction (see FIGS. 5 and 6). Herein, the term “amorphous”, as in “amorphous gel” means that the gel is only weakly diffracting X-rays in a standard powder X-ray diffraction equipment, giving patterns similar to amorphous materials, small particle sized materials or poorly crystalline materials without clearly defined diffraction peaks. It does not mean that the gel may not comprise any crystalline material.

The nanosheets are made of magnesium phosphate (with some sodium). This magnesium phosphate contains magnesium bi- and tri-phosphate. This magnesium phosphate contains hydration water. For example, it may contain between about 10 and about 20% of hydration water by weight.

A distinction should be drawn between water as a dispersing phase and hydration water. Water as a dispersing phase is the medium in which the nanosheets are dispersed. This water can be removed by drying the gel at a relatively low temperature, for example a temperature below the boiling temperature of water, such as 80° C. (See the section entitled “Water Content” in Example 1). This process will produce a product that looks and feels dry, but that still contain hydration water. Hydration water consists in molecules of water that are bonded or somehow associated with a solid (for example entrapped within it). These molecules are typically only removed from the solid by heating the solid above the boiling temperature of water, often well above this temperature, for example between 100 and 250° C. (See the section entitled “Thermogravimetry” in Example 2).

When there is no calcium in the gel, the gel may further comprise up to 200% by weight of pyrophosphate (P₂O₇⁴⁻), based on the weight of the phosphate. In embodiments, the gel may comprise between about 10% and about 20% by weight of pyrophosphate based on the weight of the phosphate. The presence of pyrophosphate makes the gel more acidic and thereby tends to improve its resistance to acidic media.

The gel may also comprise chloride (Cl⁻) ions. These may be provided by one of the compounds used for making the gel, for example calcium chloride, when it is present.

Additives can also be added to the gel. For example, these additives can aim at improving the resistance of the gel to dissolution in acidic media. Such additives include:
corn oil (for example in a concentration varying between about 0.1 and about 1.5% based on the total weigh of the gel),
sodium metaphosphate or pyrophosphate (for example in a concentration varying between about 0.125 and about 0.5% based on the total weigh of the gel),
sodium citrate (for example in a concentration varying between about 0.1 and about 10% based on the total weigh of the gel),
xantham gum (for example in a concentration varying between about 0.1 and about 1.5% based on the total weigh of the gel),
sodium alginate (for example in a concentration varying between about 0.1 and about 1.5% based on the total weigh of the gel),
carboxylate salts, such as sodium glycolate and sodium tartrate (for example in a concentration varying between about 0.1% and about 5% based on the total weigh of the gel),
carboxylic acids, such as glycolic acid and tartaric acid (for example in a concentration varying between about 0.1 and about 5% based on the total weigh of the gel), and
chitosan (for example in a concentration varying between about 0.1 and about 1.5% based on the total weigh of the gel).

The gel of the invention can be loaded with a variety of substances, including bioactive substances, depending of the desired properties and its end use. Substances that can be loaded in the gel will be discussed below when some of the end uses of the gel will be discussed.

Further, the gel can be dehydrated in an organic liquid, for example ethanol or glycerol, to partly or completely replace the water therein by these substances.

The gel of the invention can also be dried to form a xerogel. This xerogel is in embodiments, in the form of a membrane, such as a translucent membrane. Herein, “xerogels” are solids formed from the gel by drying with unhindered shrinkage. In embodiment, the drying is carried out at room temperature.

### Properties and Uses of the Magnesium Phosphate Gel

The above gel represents a new phase of phosphate minerals.

Sodium, magnesium and phosphate are all naturally found in the body. In embodiments, where they are the sole components of the gel, this indicates that the gel should be non-toxic. This would be also true of embodiments, where non-toxic substances are added to the gel.

In embodiments, this gel has a unique combination of four desirable properties: bioadhesion, thixotropy, bioresorption, and biocompatibility.

First, the inorganic gel can be thixotropic and even, in embodiments, highly thixotropic. This means that it does not flow at rest, but can reversibly liquefy with shear stress. This makes it very useful for applications requiring coating and injection. For example, in an embodiment, it can be injected through an insulin needle (φ260 μm) and solidify after injection (see Example 2—Rheology). In embodiments, the gel has a liquefaction stress of about 50 Pa or less, for example a liquefaction stress between about 30 and about 40 Pa. In embodiments, the gel has a recovery time of 10 seconds or less, for example about 6 seconds. Moreover, the gel can be injected into water without mixing or disintegrating (see Example 2—Stability in Water). In fact, in embodiments, the gel has properties similar to those of layered silicate clays.

Further, tests demonstrated that, in embodiments, this gel was bioadhesive, biocompatible and could modulate drug release (see Examples 3, 4 and 6 below). As shown in the Examples below, rheological analysis indeed revealed that the gel is thixotropic, which makes it useful for applications requiring coating and injection. The gel was tested for bioadhesion and proved adhesive to mucosa over prolonged periods of agitation. The gel also showed good biocompatibility as well as resorption. Accordingly, in embodiments, the gel makes a useful additive for minimally invasive controlled drug release applications, administered by injection.

The gel was also tested as a drug delivery system. This suggested that the gel could function as a controlled release system where control over the release rate can be obtained by modifying the degree of gel hydration.

Finally, upon drying, the gel can in embodiments form homogeneous xerogels and coatings with high specific surface area. Such xerogels, with such high surface area, are widely used as drug delivery systems for oral drug administration due to their high adsorption capacity. Such xerogels and coatings can be used for adsorbing bioactive molecules. The xerogel obtained with the gel of the invention appeared as a translucent membrane (See Example 5 below).

In addition to the previously mentioned properties, the solubility of the gel was found to be pH sensitive, and could be adjusted by modifying its ionic structure (see the addition of pyrophosphate discussed above). This property makes it an interesting material for site-specific drug delivery in inflamed tissues (low pH).

In summary, the gel of the invention is a unique inorganic gel that, in embodiments, combines several interesting properties such as stability, biocompatibility, bioresorption, bioadhesion, thixotropy, and injectability. To the best of the inventors' knowledge, these properties have never been observed in a single material before. These properties open a wide range of industrial and biomedical applications, in particular in topical, mucosal, transmucosal and injectable drug delivery applications.

The gel could be used in drug delivery systems. In particular, the gel can be used in the following areas:
mucosal topical delivery—for treating mucosal ulcerations, mucosal inflammation and periodontal diseases (for example peri-implantitis as explained below), for promoting wound healing, etc.;
transmucosal delivery—for systemic absorption of problematic drugs (large molecules) via various mucosal sites (nasal, oral, ocular, vaginal, rectal);
invasive routes of administration (subcutaneous delivery and organ-targeted delivery) for drugs (including large molecules and insoluble small molecules) and cells (including stem cells), where bioresorption and/or controlled release is desirable; and
drug-eluting medical devices (in medical and dental conditions), this could be for example a coated cardiovascular stent.

In particular, the gel could be used as a mucoadhesive for use in localized drug delivery to mucosal surfaces, more specifically to the oral mucosa. More specific examples of mucosal topical delivery include oral and dental applications (oral ulcerations, oral inflammation, periodontal diseases, etc), topical treatment of clinical manifestations on the mucosal layer of other organs such as bladder (for topical delivery of chemotherapy for bladder cancer, infections, inflammations, etc), vaginal ephitelium, ocular topical applications (keratitis, etc).

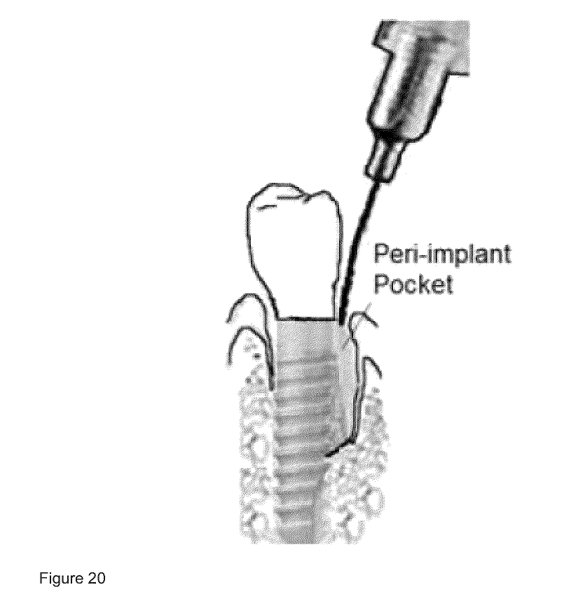

For example, the gel could be loaded with a drug, such as an antibiotic, and used as a localized drug delivery system, for example to a mucosa, in particular to the oral mucosa. This system could be used in particular for the treatment of peri-implantitis, which is a chronic infection of the bone surrounding osseointegrated dental implants. In such an application, the gel would be deposited, using a syringe or the like, in the periodontal (or peri-implant) pocket as illustrated in FIG. 20. The gel, being thixotropic would flow from the syringe into the pocket, adapt to the complex surface geometry of the dental implant, thus creating a more or less homogeneous coat, and then deliver the drug locally.

Another use in topical delivery would be in wound healing where hydrogels are known as being useful. Hydrogel dressings are seen as an essential component of wound care. They are designed to hold moisture in the surface of the wound, providing the ideal environment for cleaning the wound and also help to prevent bacteria and oxygen from reaching the wound, providing a barrier for infections. Hydrogels can be used on their own for their water absorbing and donating capacity to either absorb exudate or to hydrate the wound to promote healing. They can also incorporate drugs, in particular antimicrobials to better control wound infection and promote faster healing.

Therefore, it is to be understood that the gel can be loaded with all sorts of bioactive substances. As used herein, a bioactive substance includes any of one or more substances that produces or promotes a beneficial therapeutic, physiological, homeopathic, allopathic and/or pharmacological effect on the body. Such beneficial effects may be brought upon any animal or human patient, and various systems associated therewith, including the immune system, respiratory system, circulatory system, nervous system, digestive system, urinary system, endocrine system, muscular system, skeletal system, and the like, as well as any organs, tissues, membranes, cells, and subcellular components associated therewith. As will be appreciated by those skilled in the art, beneficial effects include assisting the more efficient functioning of the abovementioned systems, such as, for example, helping the body fight sickness and disease, helping the body to heal, etc. Exemplary bioactive substances include any element, composition or material producing a beneficial effect, including vitamins, minerals, nucleic acids, amino acids, peptides, polypeptides, proteins, genes, mutagens, antiviral agents, antibacterial agents, anti-inflammatory agents, decongestants, histamines, anti-histamines, anti-allergens, allergy-relief substances, homeopathic substances, pharmaceutical substances (i.e. a drug), such as antibiotics and other drugs, and the like.

The gel may also comprise additives like those usually found in other compositions with the same end use. For example, for composition for oral mucosal delivery, the gel can comprise flavoring, oral-hygiene agents, colorants and/or opacifying agents.

### Method of Making the Magnesium Phosphate Gel

There is also provided a method of producing a magnesium phosphate gel. The method comprise the step of providing (A) a aqueous solution comprising sodium hydroxide (NaOH) and (B) a aqueous solution comprising phosphoric acid (H₃PO₄) or monomagnesium phosphate (Mg(H₂PO₄)₂). Then, magnesium hydroxide (Mg(OH)₂) or trimagnesium phosphate (Mg₃(PO₄)₂), and optionally calcium chloride or calcium hydroxide, is dissolved in the phosphoric acid or monomagnesium phosphate containing solution. Finally, this last solution is mixed with the solution comprising sodium hydroxide (NaOH). The gel forms within seconds of mixing both solutions. For better results, the time between the addition of the magnesium hydroxide or trimagnesium phosphate and the addition of the sodium hydroxide solution should be no more than several minutes, for example 10 minutes.

The concentration and quantity of solutions and solutes used to make the gel will be chosen so that the quantity of phosphate, magnesium (and optional calcium), and sodium in the gel respects the mole fractions and the water content discussed in the previous section.

In embodiments, the solution of phosphoric acid (H₃PO₄) or monomagnesium phosphate (Mg(H₂PO₄)₂) in water comprises phosphoric acid. In embodiments, magnesium hydroxide is dissolved into this dissolution.

All the above steps can advantageously be carried out at room temperature.

### DEFINITIONS

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

Herein, the term “about” has its ordinary meaning. For example, it may means plus or minus 10% of the numerical value thus qualified.

Unless otherwise defined, all technical and scientific terms used herein have the same meaning as commonly understood by one of ordinary skill in the art to which this invention belongs.

Other objects, advantages and features of the present invention will become more apparent upon reading of the following non-restrictive description of specific embodiments thereof, given by way of example only with reference to the accompanying drawings.

### DESCRIPTION OF ILLUSTRATIVE EMBODIMENTS

The present invention is illustrated in further details by the following non-limiting examples.

### Example 1

### Formation of Gels

Gels were made by dissolving magnesium hydroxide (Mg(OH)₂) in a phosphoric acid (H₃PO₄) aqueous solution, reacting the obtained mixture with sodium hydroxide (NaOH) in solution, and filtering the product. A whole range of concentration of these reactants was tested. Gels were only obtained in a specific window of concentrations. Not all concentration combinations allow forming gels; some formulations precipitated crystals, some precipitated nothing, and some formulations were not acidic enough to dissolve the magnesium hydroxide in the first place.

Table 1 shows the different products obtained for an array of reactant concentrations. In these experiments, 25 mL of phosphoric acid (1M-0.5M) was used. Magnesium hydroxide was then dissolved in the acid to a concentration of 0.2M-0.1M. Then, 25 mL of sodium hydroxide at 1.0M-0.2M was added to the mixture.

TABLE 1

| | | | | | |
|---|---|---|---|---|---|
| [NaOH] in mol/L | 1.0 | 0.8 | 0.6 | 0.4 | 0.2 |

[Mg(OH)₂] = 0.2 mol/L

| | | | | | | |
|---|---|---|---|---|---|---|
| [H₃PO₄] | 1.00 | GEL(thick) | crystal | crystal | crystal | crystal |
| in mol/L | 0.75 | undis- | undis- | undis- | undis- | undis- |
| | | solved | solved | solved | solved | solved |
| | 0.50 | undis- | undis- | undis- | undis- | undis- |
| | | solved | solved | solved | solved | solved |

[Mg(OH)₂] = 0.15 mol/L

| | | | | | | |
|---|---|---|---|---|---|---|
| [H₃PO₄] | 1.00 | crystal | crystal | crystal | crystal | nothing |
| in mol/L | 0.75 | GEL | crystal | crystal | crystal | crystal |
| | 0.50 | undis- | undis- | undis- | undis- | undis- |
| | | solved | solved | solved | solved | solved |

[Mg(OH)₂] = 0.1 mol/L

| | | | | | | |
|---|---|---|---|---|---|---|
| [H₃PO₄] | 1.00 | crystal | crystal | crystal | nothing | nothing |
| in mol/L | 0.75 | GEL | crystal | crystal | crystal | undis- |
| | | | | | | solved |
| | 0.50 | GEL(thick) | GEL | GEL | undis- | undis- |
| | | | | | solved | solved |

In the above table, the mention “GEL” indicates a gel that is grey, translucent, fairly soft, and thixotropic. In contrast, the mention “GEL(thick)” means gels that are very white, opaque and thick. The present invention is concerned with the products marked “GEL” only.

In some cases, the formation of the gel was sensitive to the time taken to mix the reactants. Once the magnesium hydroxide was dissolved in the phosphoric acid, it could not be left out for more than several minutes. Sodium hydroxide had to be mixed in and the gel formed. Otherwise, the gel might crystallize. The different formulations were not equally sensitive to this factor, some were more affected, others less so.

Some of the tests below were carried on a gel formed using 0.75M phosphoric acid in which magnesium hydroxide is dissolved to 0.15M and to which a 1M solution of sodium hydroxide is added in equal proportions. This gel formulation will be referred hereinafter as “0.75 0.15 1”. A similar nomenclature will be adopted for the other gel formulations.

The 0.75 0.15 1 is not very sensitive to the time taken to prepare it, produces a large amount of thixotropic gel with an interesting texture.

### Water Content

Even after filtering, the gels obtained were composed largely of water. Heated at 80° C. for 24 hours, they lost over 90% of their mass. Although the exact mass fraction of water differed slightly between different gel formulations, it was always between 0.92-0.96.

Once a gel was filtered, it still acted like a gel even if it was then diluted with water. If the gel was diluted up to a fraction, i.e. (gel+water)/(gel), of 1.3, it was still a gel. At a fraction of around 1.35-1.4, the gel became very thin, but was still thixotropic. For example, if it was left standing for 30 seconds, the container in which it resided could be turned upside down without the gel flowing, however with one shake of the container, the gel turned into a runny liquid. At a dilution fraction of about 1.5-1.6 or more, the gel lost its thixotropic properties and behaved like a liquid.

### PH

The pH of the gels was affected by the concentration of the reactants. This was expected as phosphoric acid is an acid and sodium hydroxide is a base.

For example, gel 0.5 0.1 1 is basic because the initial concentration of acid is only 0.5M while the concentration of the base is 1M. Likewise, 1 0.2 1 is neutral because 1M acid is mixed with 1M base.

Gel 0.75 0.15 1 is a basic gel with a final pH of approximately 10.75.

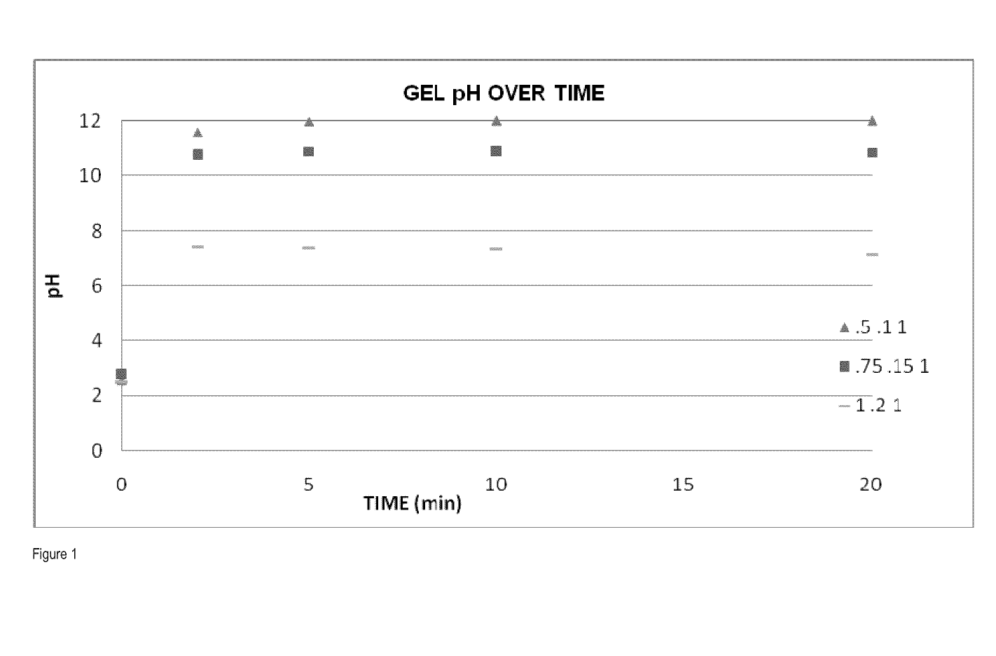

FIG. 1 shows the pH after formation for three gel formulations. In these experiments, 25 mL of phosphoric acid at 1M-0.5M were used. Magnesium hydroxide was then dissolved in the acid to a concentration of 0.2M-0.1M. Then, 25 mL of sodium hydroxide at 1.0M was added to form a gel. At t=0, the pH reading is that of the phosphoric acid with the magnesium hydroxide dissolved in it. Then, the sodium hydroxide was added and pH readings of the gel were taken at t=2 min, 5 min, 10 min, and 20 min.

### Large Scale Mixtures

Most of the above gels were made in small volume batches of 50 mL. However, no difficulties were encountered when making ×10 scale batches. Gel formulations 1 0.2 1; 0.75 0.15 1; 0.5 0.1 0.8; and 0.25 0.05 0.8 were made at 500 mL.

### Modified Gels

Some of the reactants used for making the gels were replaced by similar chemicals.

### Calcium Hydroxide Instead of Magnesium Hydroxide

Gels 1 0.2 1; 0.75 0.15 1; and 0.5 0.1 1 were made by replacing 10% (by weight) of the magnesium hydroxide by calcium hydroxide (Ca(OH)₂). Although, calcium hydroxide was somewhat more difficult to dissolve than the magnesium hydroxide, gels formed normally. These gels were slightly more alkaline than the pure-magnesium gels.

### Calcium Chloride Instead of Magnesium Hydroxide

In gel 0.75 0.15 1, 10 to 100% (in increments of 10%) of the magnesium hydroxide was replaced by calcium chloride (CaCl₂). Calcium chloride dissolved very well. Formulations with 10-70% of the magnesium hydroxide replaced produced gels. In particular, formulations with 10-30% produced good quality gels. Replacing more magnesium hydroxide (80-100%) yielded crystals.

### Potassium Hydroxide Instead of Magnesium Hydroxide

When sodium hydroxide was replaced completely with potassium hydroxide (KOH), the products were crystalline.

### Pyrophosphoric Acid in Addition to Phosphoric Acid

Pyrophosphoric acid as a solid was added to the phosphoric acid. Pyrophosphoric acid dissolved readily. 1% (by weight) additions of pyrophosphoric acid to gels 1 0.2 1; 0.75 0.15 1; and 0.5 0.1 1 did not affect normal formation of the gels. However, this changed when the addition was increased to 10% weight. TABLE 2 shows the products of an array of gel formulations with 10% pyrophosphoric acid as an additive. In these experiments, 25 mL of phosphoric acid at 1M-0.5M was used. Solid pyrophosphoric acid was added to the phosphoric acid at 10% weight, which represented 2.5 g. Magnesium hydroxide was then dissolved in the acid at a concentration of 0.4M-0.2M. Then, 25 mL of sodium hydroxide at 1.0M was added to the mixture.

TABLE 2

| [Mg(OH)₂] in mol/L | | 0.4 | 0.3 | 0.2 |
|---|---|---|---|---|
| [NaOH] in mol/L | | 1 | 1 | 1 |
| [H₃PO₄] in mol/L | 1 | GEL(thick) | GEL(thick) | nothing |
| | .75 | undissolved | GEL(thick) | nothing |
| | .5 | undissolved | GEL(thick) | GEL |

Pyrophosphoric acid is a very strong acid. It thus made the solution of phosphoric acid and magnesium hydroxide much more acidic than it would otherwise be. This meant that more magnesium hydroxide could be dissolved. In fact, the maximum concentration of magnesium hydroxide was 0.4M with pyrophosphoric acid compared to 0.2M without it. The increased initial acidity also meant that the gels formed were less alkaline. These gels indeed had pHs between 3.5 and 4.25.

Pyrophosphoric acid was added by mass as high as 50% at which point a hard white gel formed at high concentrations of magnesium hydroxide.

### Pyrophosphoric Acid Instead of Phosphoric Acid

When pyrophosphoric acid replaced phosphoric acid in a typical gel such as 0.75 0.15 1, no product formed. However when the amount of magnesium hydroxide was increased to 0.3M, the pyrophosphoric acid gel 0.75 0.15 1 was a thick white gel.

### Others

10% pyrophosphoric acid was also added when making gels with 10-30% calcium chloride replacing the magnesium hydroxide. The products were thick white gels.

### X-Ray Diffraction

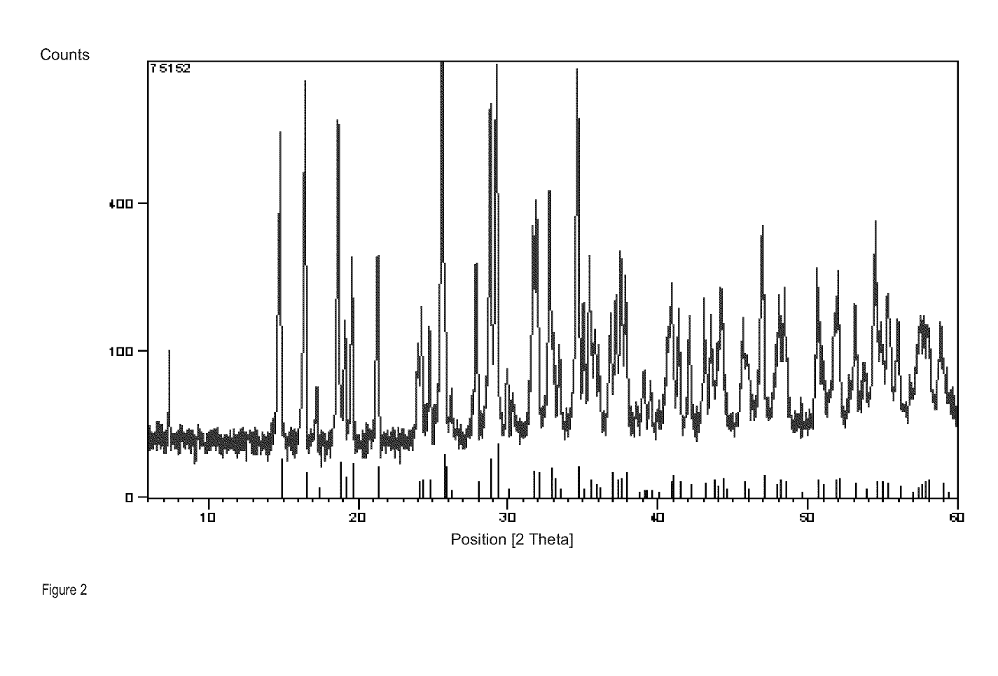

X-ray diffractograms of gels 1 0.2 1 and 0.5 0.1 1 and of the crystal products resulting of formulations 0.75 0.15 0.2; 0.5 0.1 0.8, and 0.25 0.05 0.4 were recorded. FIGS. 2-6 show these diffractograms.

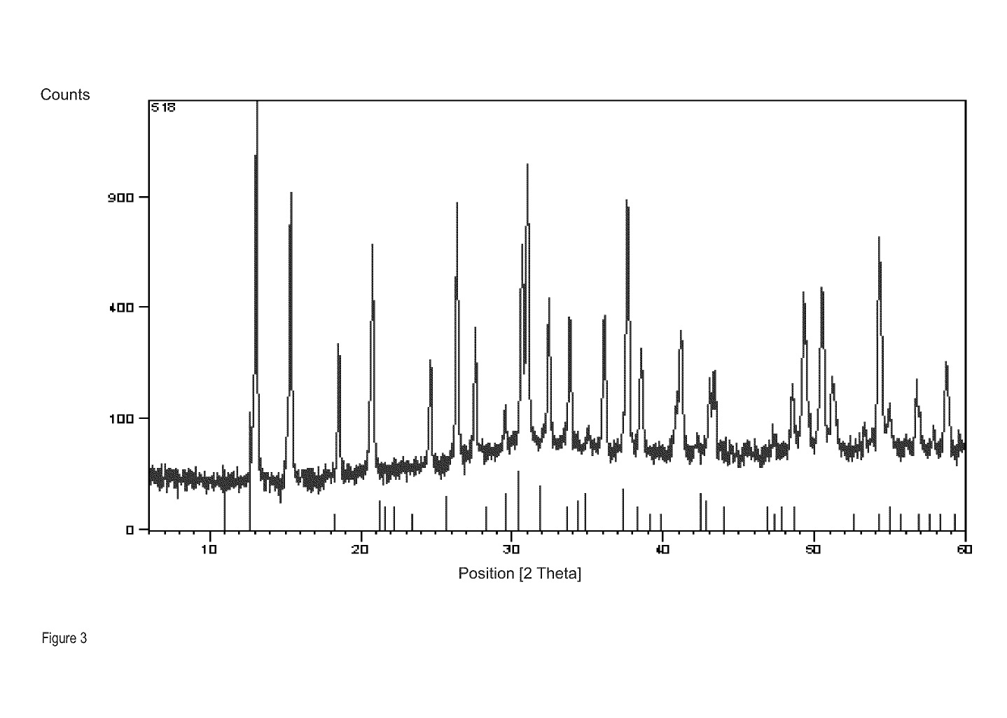

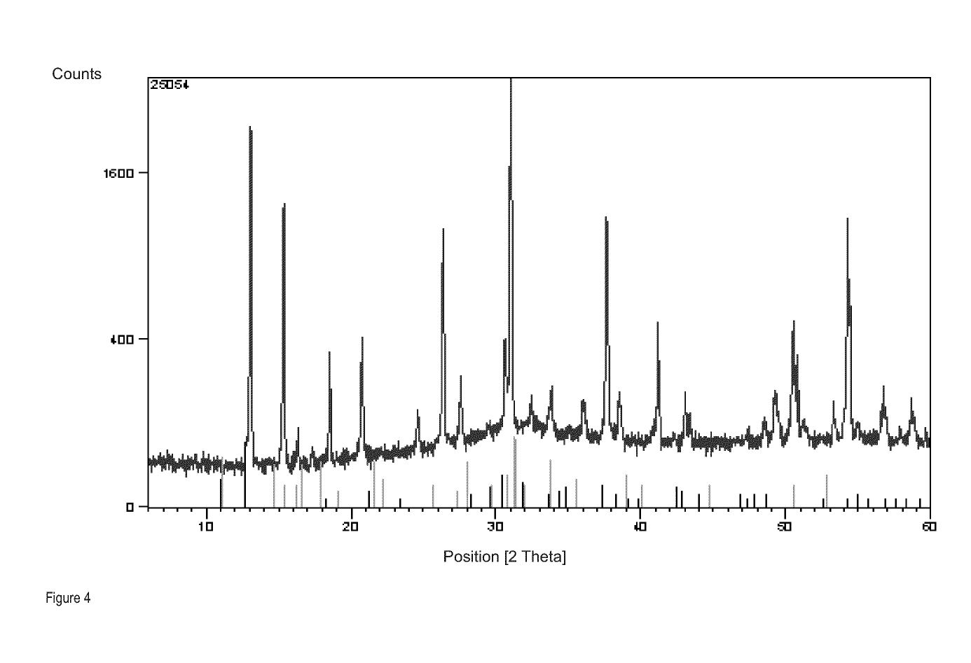

The crystal products were identified as forms of magnesium phosphates. More specifically, the crystal product of 0.75 0.15 0.2 appear to contain Newberyite (MgHPO₄:3H₂O) as shown in FIG. 2. The crystal product of 0.5 0.1 0.8 appear to contain Bobierrite (Mg₂(PO₄)₂:8H₂O) as shown in FIG. 3. The crystal product of 0.25 0.05 0.4 appear to contain a mixture of Bobierrite (Mg₂(PO₄)₂:8H₂O) and magnesium phosphate hydrate (Mg₂(PO₄)₂:22H₂O) as shown in FIG. 4. On the other hand, the gels appeared to be mostly amorphous. Gel 0.5 0.1 1 had a very weak X-ray diffraction pattern, possibly indicating an amorphous structure, as shown in FIG. 5. Gel 1 0.2 1 also had a weak X-ray diffraction pattern as shown in FIG. 6. It however nevertheless contained some crystalline peaks, indicating an amorphous structure with some crystalline content.

### Acid Stability

The gels did not dissolve or disintegrate when submerged in water.

When placed in an acidic solution, the gels dissolved over 24 hours. Many additives were tested to deter the gels from dissolving in an acidic solution. Table 3 shows how gels dissolved over 24 hours with different additives.

The acidic solutions used were sodium citrate/citric acid buffers. All experiments were done with 0.2 mL of gel. The corn oil, sodium metaphosphate, sodium pyrophosphate, sodium citrate, xanthan gum, sodium alginate, and chitosan solutions were prepared by taking a 0.5-0.125% (by weight) solution of the additive, mixing it with an equal volume of gel, and filtering the solution. The calcium chloride gels were made by replacing 10-30% of the magnesium hydroxide with calcium chloride in the actual production of the gel. The pyrophosphoric acid gels were prepared by adding 10% (weight) pyrophosphoric acid to the phosphoric acid before adding the magnesium hydroxide when producing the gels. And the ethanol and glycerol gels were made by dehydrating the gel in a solution of ethanol or glycerol.

TABLE 3

| | | Mass % of Gel Remaining | | | |
|---|---|---|---|---|---|
| | | After 24 Hours (%) | | | |
| Additive | Gel | pH 3 | pH 4 | pH 5 | pH 6 |
| None | .75 .15 1 | 0 | 33 | 75 | 90 |
| 0.5% Corn Oil | .75 .15 1 | 25 | 50 | 75 | 100 |
| 0.5% Sodium Metaphosphate | .75 .15 1 | 25 | 50 | 66 | 75 |
| 0.5% Sodium Pyrophosphate | .75 .15 1 | 25 | 33 | 66 | 90 |
| 0.25% Sodium Pyrophosphate | .75 .15 1 | 33 | 50 | 80 | 100 |
| 0.125% Sodium Pyrophosphate | .75 .15 1 | 33 | 50 | 80 | 100 |
| 0.5% Sodium Citrate | .75 .15 1 | 0 | 25 | 75 | 100 |
| 10% CaCl₂ | .75 .15 1 | 25 | 50 | 75 | 90 |
| 20% CaCl₂ | .75 .15 1 | 25 | 50 | 75 | 90 |
| 30% CaCl₂ | .75 .15 1 | 50 | 60 | 80 | 100 |
| 0.5% Xanthan Gum | .75 .15 1 | 50 | 70 | 90 | 100 |
| 0.5% Sodium Alginate | .75 .15 1 | 50 | 60 | 80 | 100 |
| 0.5% Chitosan | .75 .15 1 | 25 | 50 | 75 | 100 |
| Ethanol | .75 .15 1 | 0 | 0 | 0 | 0 |
| Glycerol | .75 .15 1 | 0 | 0 | 0 | 0 |
| 10% Pyrophosphoric Acid | 1 .4 1 | 100 | 100 | 100 | 100 |
| 10% Pyrophosphoric Acid | 1 .4 1 | 100 | 100 | 100 | 100 |
| 10% Pyrophosphoric Acid | .5 .3 1 | 100 | 100 | 100 | 100 |

### Dehydration of Gel

The gels are over 90% water.

Dehydration of the gel involved removing that water and replacing it with ethanol and glycerol. Two pieces of gel (2 mL each) were placed in 20% ethanol and 20% glycerol solution. Every hour each beaker was drained of the solution and replaced with a 10% stronger solution. After 9 hours, the gels were finally placed in a 100% ethanol and 100% glycerol solution. At the end of this process, each gel had been drained of water and had absorbed its respective solution.

### Example 2

### Materials

The following reagents were purchased from Sigma-Aldrich and used without further purification: magnesium hydroxide (MO), sodium hydroxide (SH) and phosphoric acid (PA).

### Methods

The precipitation of magnesium phosphates in the presence of sodium ions was studied by dissolving magnesium hydroxide (300-0 mg) into to a 10 ml solution of phosphoric acid (1.0-0.0 M) and sodium hydroxide (1.0-0.0 M).

The magnesium hydroxide powder was first added to the phosphoric acid solution and mixed until it was dissolved. Then, sodium hydroxide (as a solution) was added to the mixture. Several batches representing different MO:PA:SH ratios were prepared.

The pH of the resulting solutions was measured, and the precipitates were washed and dried for analysis with EDX, transmission electron microscopy (TEM) and BET surface area analysis.

Phase composition of the precipitates was characterized with X-ray diffraction (XRD).

A vertical-goniometer X-ray diffractometer (Philips model PW1710, Bedrijven b. v. S&I, The Netherlands), equipped with a Cu Kα radiation source, was used for the powder diffraction pattern collection. Data was collected from 20° to 40° with a step size of 0.02° and a normalized count time of 1 s per step. The phase composition was examined by means of the International Centre for Diffraction Data (ICDD) reference patterns.

The gel sample was tested for rheological properties with a rheometer Rheostress I (Haake, Thermo) with two 20.0 mm parallel plates with a gap of 0.2 mm at 37° C.

### Gel Formation

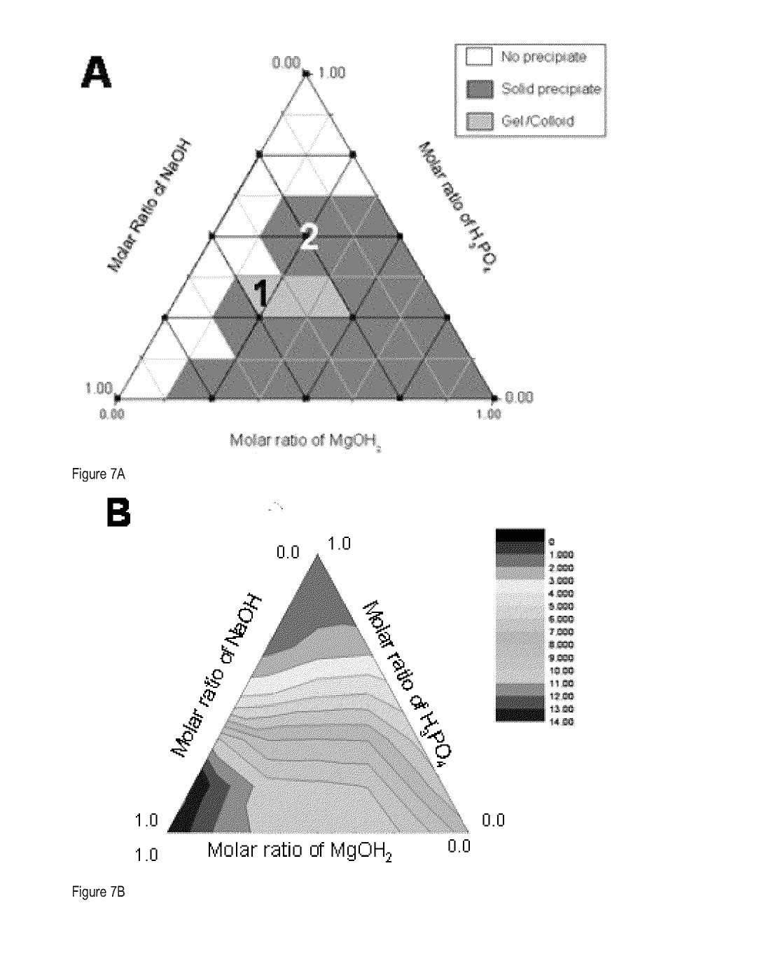

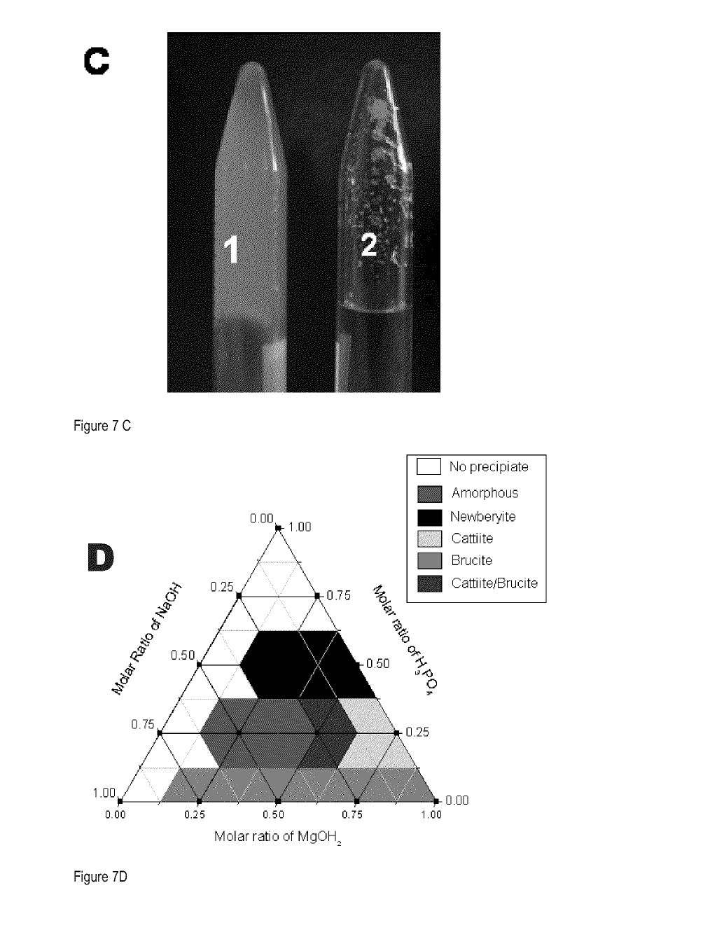

The solutions where any one of magnesium, sodium, or phosphate was absent did not form gels. The solutions with more than 0.75 M of phosphoric acids did not form precipitates either. Solutions with magnesium hydroxide (0.25 M), phosphoric acid (0.4-0.5M), and sodium hydroxide (0.5-0.6 M) formed an amorphous gel. The remaining solutions precipitated to form crystals (see FIGS. 7A and C).

FIG. 7A is an approximate phase diagram presenting the nature of the precipitates obtained from sodium/phosphate/magnesium solutions. Darker gray indicate the region where precipitation occurs. The region where there is neither gel, nor precipitate is white. The approximate concentration region where gels form is pale gray and contains the label “1”.

FIG. 7B is an interpolation diagram showing the pH of the different solutions as a function of the various components concentration. As discussed above, the gels are neutral or basic.

FIG. 7C is a photographic images of the gel labeled “1” in FIG. 7A (i.e. H₃PO₄:Mg(OH)₂:NaOH molar ratio of 0.25:0.25:0.50) and the crystalline precipitate labeled “2” in FIG. 7A (i.e. H₃PO₄:Mg(OH)₂:NaOH molar ratio of 0.50:0.25:0.25).

FIG. 7D is a phase diagram of summarizing the XRD findings for the different solutions. It can be seen that the precipitates are crystalline in nature and that the region where gels are obtained is contained within an amorphous region.

### Stability in Water

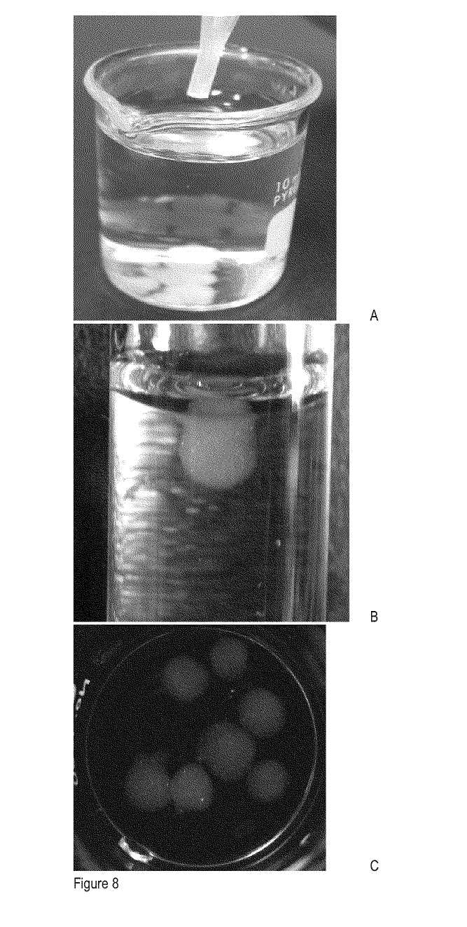

Despite their high water content, the magnesium sodium phosphate gels obtained were stable in water, and could be injected into distilled water to form pellets. FIG. 8 shows photographs in which a gel prepared by mixing 75 mg of Mg(OH)₂ into a 10 ml solution of 0.75M SH and 0.5 PA. The pictures shows the pipetting of the gel into a beaker of distilled water (A), a droplet of the gel form a cohesive sphere after being pipetted drop wise into distilled water (B), and seven gel pellets on the bottom a 20 ml beaker filled with distilled water (C).

### X-Ray Diffraction (XRD)

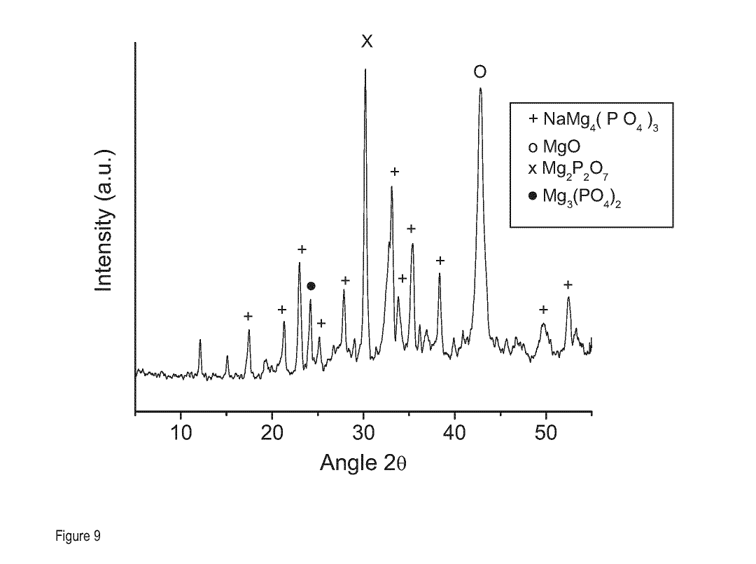

Using X-ray diffraction analysis, the gel 0.75 0.15 1 appeared to be amorphous. Other precipitates obtained were Newberyite, Cattiite, Brucite (magnesium hydroxide), and mixtures of Cattiite with Brucite (see FIG. 7D). The amorphous gel was heated to 700° C. (i.e. calcined) and XRD analysis was performed on the heated gel to characterize its composition. The XRD pattern obtained (shown in FIG. 9) matched that of magnesium pyrophosphate, trimagnesium phosphate, and sodium magnesium phosphate. This indicates that the gel is probably composed of tri-phosphate, di-phosphate, magnesium, and sodium ions.

### Elemental Composition

To characterize the elemental composition of the solid and liquid phases of the gel, EDX analysis was performed on the gel 0.75 0.15 1 either (A) filtered-washed and dried (in vacuum at 40° C.) or (B) dried without filter-washing. The elemental composition of the washed and un-washed gels (as atomic percentage values) is presented in Table 4.

TABLE 4

| Atomic | Unwashed- | Standard | Washed- | Standard |
|---|---|---|---|---|
| % | dried | Deviation | dried | Deviation |
| O | 62.29 | 2.22114 | 68.0925 | 1.93272 |
| Na | 20.4475 | 3.05186 | 2.4425 | 0.09708 |
| Mg | 3.33 | 1.57067 | 16.215 | 0.22128 |
| P | 13.9275 | 0.76991 | 13.32 | 1.76206 |

The un-washed gel had a high concentration of sodium phosphate, whereas the washed gel was composed of sodium magnesium phosphate. The ratio between magnesium and phosphate ions in the washed samples is 2.62, which indicates the presence of both di-magnesium and tri-magnesium phosphate species in the structure.

### Thermogravimetry

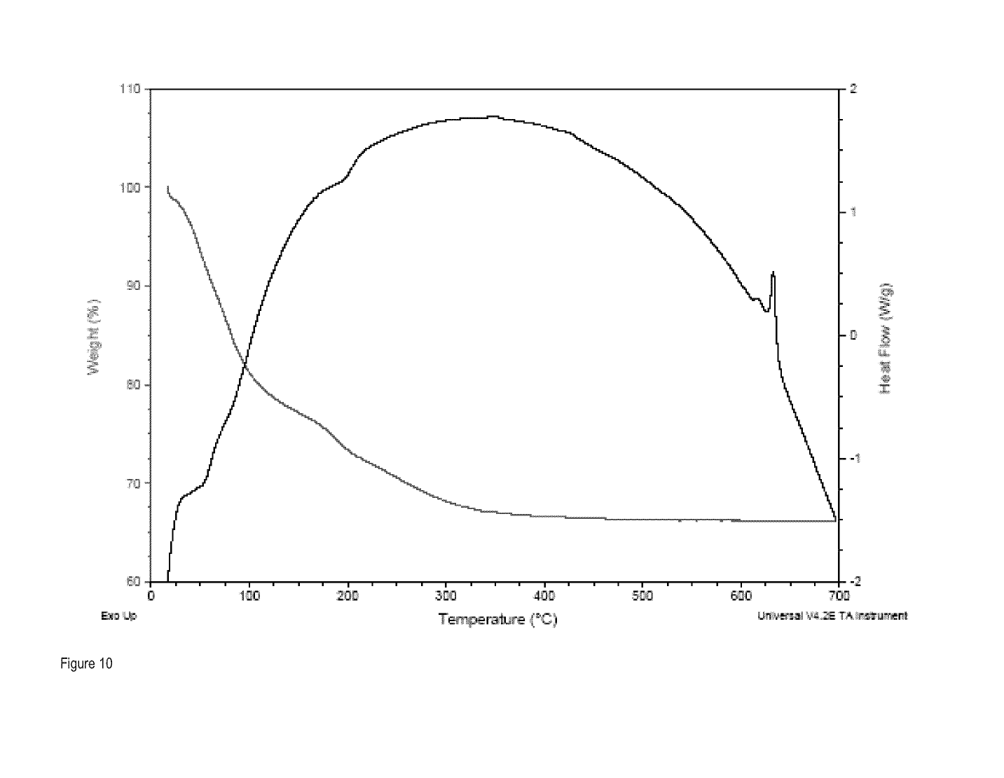

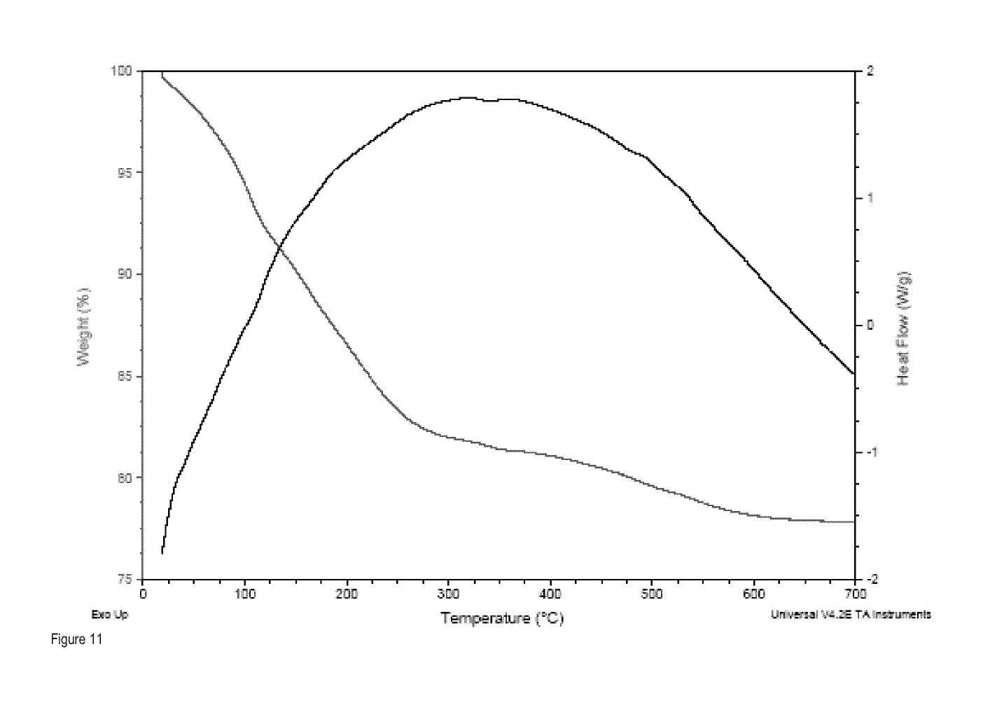

Thermogravimetry analysis (TGA) and differentials scanning calorimetric (DSC) analysis of washed and un-washed dried-gel samples were performed (see FIGS. 10 and 11, where the DSC analyses are the bell shape curves). The percentage of weight loss between 100° C. and 250° C. was of ˜16% for the un-washed samples, and ˜10% for the washed gel. These results confirm the presence of large amounts of hydration water within the solid structure of the gel. Minor crystallization exothermic peaks were detected between 300 and 500° C., suggesting crystallization of HPO₄ into pyrophosphate. This confirms the presence of small amounts of HPO₄ groups in the gel.

### FTIR

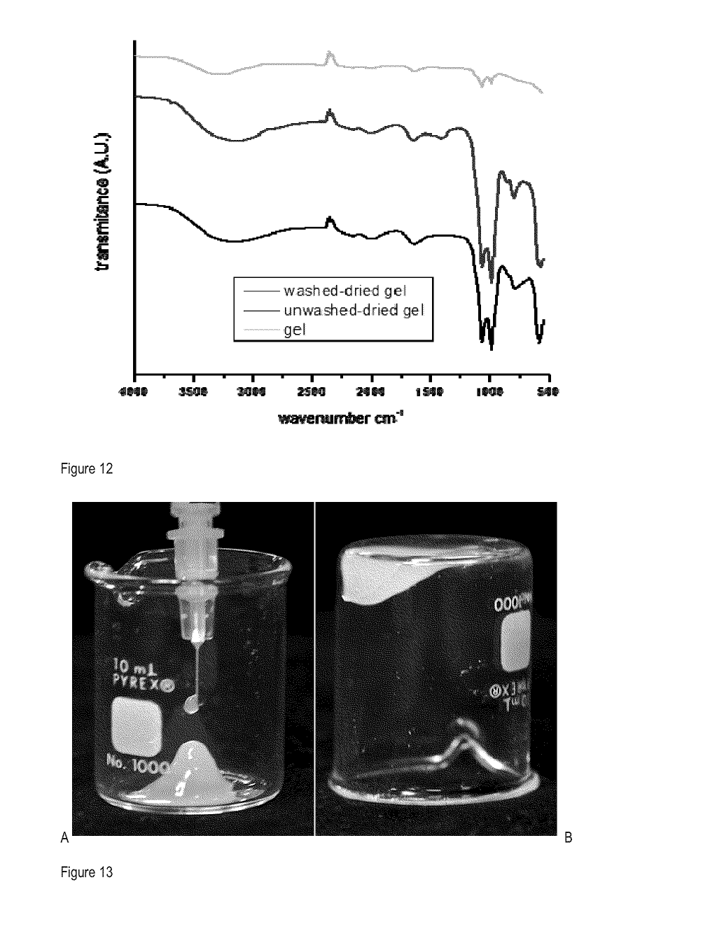

Infrared spectroscopy was performed to characterize the chemical composition of the gels. FIG. 12 shows the infrared spectra of the hydrated gel (top) and the unwashed (middle) and washed (bottom) dried gel samples.

Very strong peaks at 980 cm⁻¹ and 1062 cm⁻¹ indicating PO stretching could be observed in all the samples. In addition, bands characteristic of di-phosphate groups (P—O(H)) were also detected in the dried-unwashed gel and in the washed-dried gel samples at 858 cm⁻¹ and 1900-2100 cm⁻¹. Also, bands characteristic of hydration water were observed at 1648 cm⁻¹ and 2900-3400 cm⁻¹. A band characteristic of Na₂HPO₄ was observed in the unwashed-dried samples at 1402 cm⁻¹.

### Rheology

Rheological analysis revealed that the gel 0.75 0.15 1 had an extreme thixotropic behavior. FIG. 13 shows photographs where the gel is injected through an insulin needle (A) and then recovering (B).

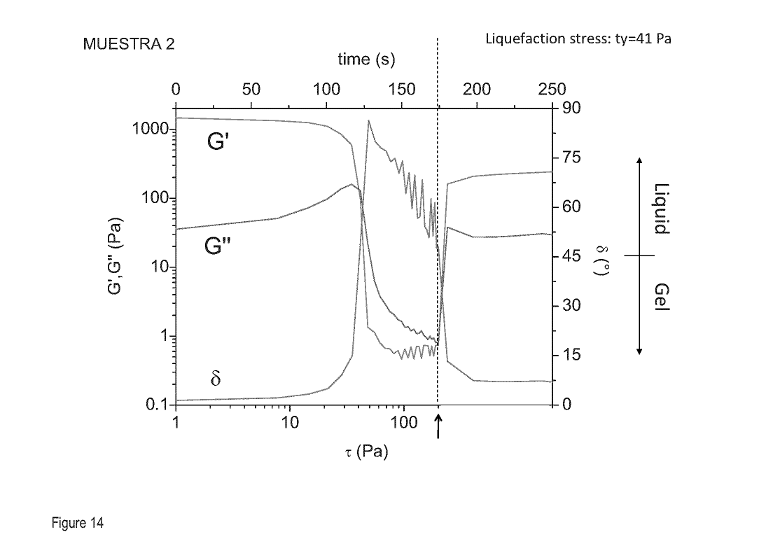

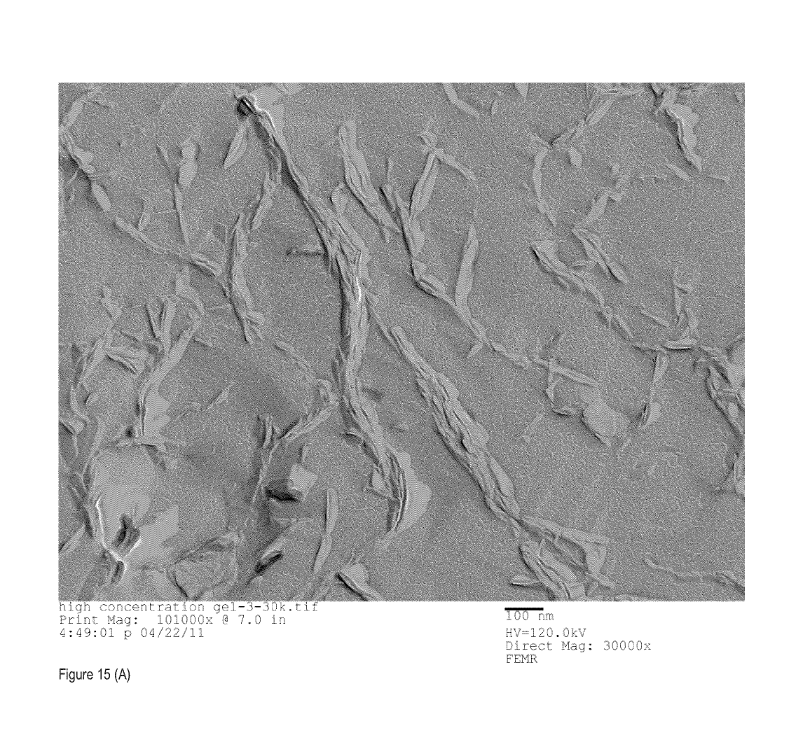

FIG. 14 shows the results of the rheological analysis of the gel. The liquefaction stress was very low (40-30 Pa) and the recovery time was very short (˜6 seconds). In other words, the gel-to-liquid and liquid-to-gel transitions occur within less than 6 seconds of the induction and removal of shear stress. To the inventors' knowledge, this extremely high speed of transition is very uncommon in hydrogels, and unheard of in any other biomaterials.

### Ultrastructure

Upon TEM analysis, the gel 0.75 0.15 1 appeared to be formed of nanosheets that are about 200 nm wide, very thin and up 1 μm long. These nanosheets appeared crystalline when observed by TEM, although the gel itself appeared amorphous when studied by X-ray diffraction. The nanosheets in the original hydrated gel however appeared amorphous when studied by electron diffraction.

The dried gel had a BET specific surface area of BET 59.2087 m²/g and a density of 0.1527±0.0078 g/ml. FIG. 15 is crio-TEM images showing the gel ultrastructure.

### Conclusion

The above characterization of the sodium magnesium phosphate gel indicates that this material is composed of flat layered nano-crystals that are hydrated, and are composed of a mixture of di- and tri-phosphate ions combined with magnesium, and small amounts of sodium.

### Example 3

### Bioadhesion

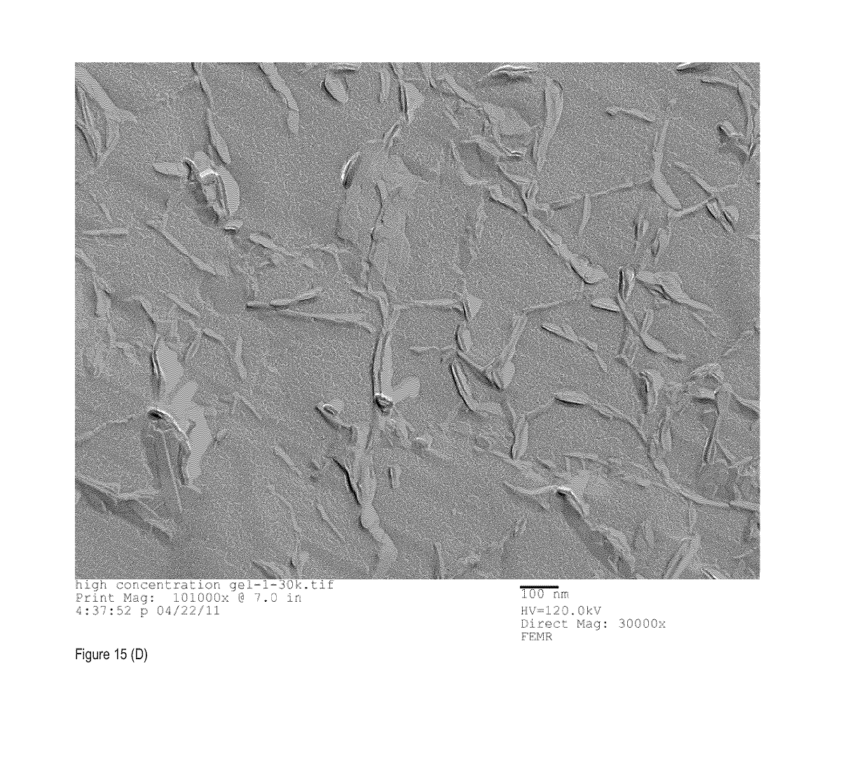

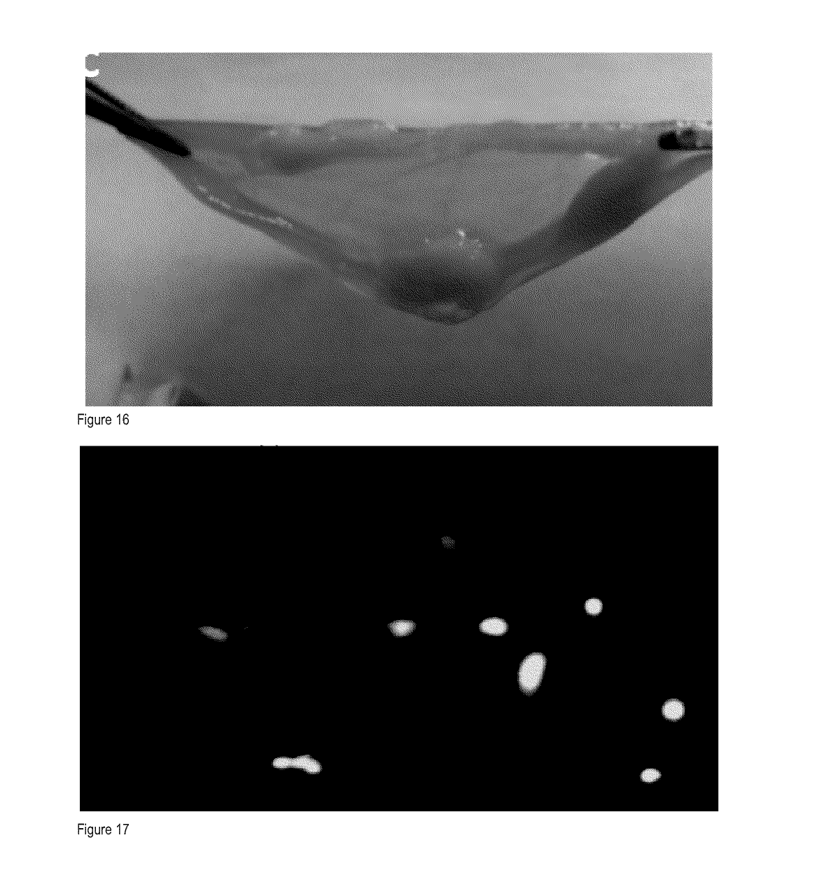

Gel 0.75 0.15 1 was tested for bioadhesion on explanted gastric mucosae. It proved highly adhesive to fresh gastric mucosa from a sacrificed rabbit over prolonged periods of agitation. FIG. 16 is a photograph showing the gel adhered to gastric mucosa after 24 hours of incubation in aqueous oscillating medium.

### Example 4

### Biocompatibility

Gel 0.75 0.15 1 was also tested for cellular biocompatibility by cultivating human bone marrow stem cells into it, and tested for cytotoxicity. The test revealed over 50% cell survival within 24 hours, indicating good biocompatibility (see FIG. 17).

Gel 0.75 0.15 1 was injected intramuscularly and subcutaneously in mice without causing any ill effects. Five days after injection, the animals were sacrificed. Histopathological examination revealed the gel had partially resorbed without causing any major inflammation at the injection site.

### Example 5

### Xerogels

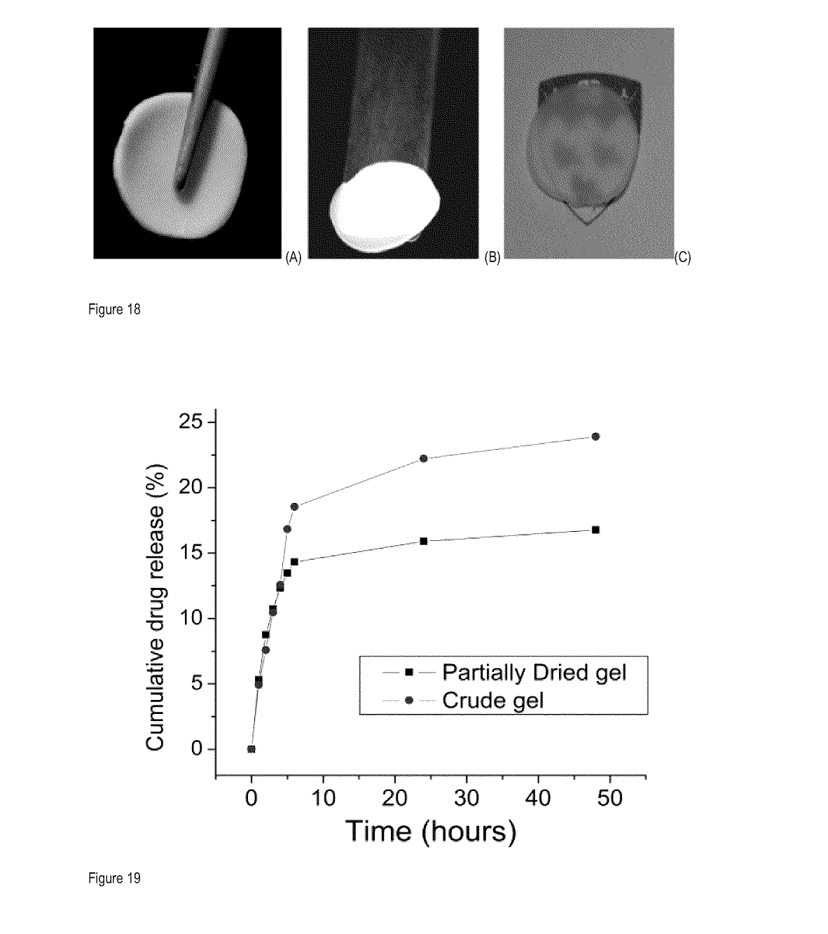

Gel 0.75 0.15 1 was dried into a xerogel forming translucent membranes that have a specific surface area of ˜60 m²/g and a density of 0.15 g/cm³. FIGS. 18 (A) to (C) show this process.

### Example 6

### Drug Delivery

Gel 0.75 0.15 1 was tested as a drug delivery system in two forms: totally hydrated (crude), and partially hydrated (dried by filtration). FIG. 19 shows the release profile of diclofenac (a model drug) from fresh and dried gel as a function of time. The slower drug release rate from the partially dried gel compared to the crude one suggests the gel can function as a controlled release system where control over the release rate can be obtained by modifying the degree of gel hydration.

The scope of the claims should not be limited by the preferred embodiments set forth in the above examples, but should rather be given the broadest possible interpretation consistent with the description as a whole.

### REFERENCES

The present description refers, above and below, to a number of documents, the content of which is herein incorporated by reference in their entirety.
US 2003/0175217;
WO 2011/126537;
Franciele Viana Fabri, Rogério Rodrigues Cupertino, Mirian Marubayashi Hidalgo, Rúbia Maria Monteiro Weffort de Oliveira, and Marcos Luciano Bruschi: Preparation and characterization of bioadhesive systems containing propolis or sildenafil for dental pulp protection, Drug Development and Industrial Pharmacy, 2011; 37(12): 1446-1454;
Needleman I G, Martin G P, Smales F C: Characterisation of bioadhesives for periodontal and oral mucosal drug delivery. J Clin Periodontol 1998: 25: 74-82;
David s. Jones, A. David Woolfson, and Andrew F. Brown: Textural Analysis and Flow Rheometry of Novel, Bioadhesive Antimicrobial Oral Gels, Pharmaceutical Research, Vol. 14, No. 4, 1997 and
K. Pal, A. K. Banthia and D. K. Majumdar: Polymeric Hydrogels: Characterization and Biomedical Applications—A mini review, Designed Monomers and Polymers 12 (2009), Pages 197-200.

1. A magnesium phosphate gel comprising water as a dispersing phase and phosphate ions (PO₄³⁻), a divalent cation, and sodium ions (Na⁺), wherein the divalent cation is magnesium (Mg²⁺) or a mixture of magnesium and calcium (Ca²⁺), the mixture comprising up to 30% by weight of calcium based on the total weight of the mixture.

2. The magnesium phosphate gel of claim 1, comprising the phosphate ions, the divalent cation and sodium ions at mole fractions of about 0.33 to about 0.44, about 0.03 to about 0.09, and about 0.48 to about 0.63, respectively.

3. The magnesium phosphate gel of claim 2, comprising the phosphate ions, the divalent cation and sodium ions at mole fractions of about 0.36 to about 0.42, about 0.05 to about 0.08, and about 0.50 to about 0.57, respectively.

4. The gel of any one of claims 1 to 3, comprising more than about 50% by weight of water as the dispersing phase based on the total weight of the gel.

5. The gel of claim 4, comprising more than about 70% by weight of water as the dispersing phase based on the total weight of the gel.

6. The gel of claim 5, comprising more than about 90% by weight of water as the dispersing phase based on the total weight of the gel.

7. The gel of claim 6, comprising between about 92% and about 98% by weight of water as the dispersing phase based on the total weight of the gel.

8. The gel of claim 7, comprising between about 92% and about 96% by weight of water as the dispersing phase based on the total weight of the gel.

9. The gel of any one of claims 1 to 8, wherein the phosphate, magnesium, optional calcium, and sodium ions form nanosheets.

10. The gel of claim 9, wherein the nanosheets are about 200 nm wide, about 10 nm thick and up to about 1 μm long.

11. The gel of claim 9 or 10, wherein the nanosheets contain magnesium phosphate.

12. The gel of claim 11, wherein the nanosheets contain magnesium bi- and tri-phosphate.

13. The gel of any one of claims 9 to 12, wherein the nanosheets further contain hydration water.

14. The gel of claim 13, wherein the nanosheets contain from about 10 to about 20% by weight of hydration water based on the water of the dried gel.

15. The gel of any one of claims 1 to 14, wherein the divalent cation is the mixture of magnesium and calcium.

16. The gel of claim 15, wherein the mixture comprise between about 10% and about 30% by weight of the calcium based on the total weight of the mixture.

17. The gel of any one of claims 1 to 14, wherein the divalent cation is magnesium.

18. The gel of claim 17, comprising phosphate, magnesium, and sodium ions at mole fractions of about 0.39, about 0.08, and about 0.53, respectively

19. The gel of claim 17 or 18, further comprising up to about 200% by weight of pyrophosphate (P₂O₇)⁴⁻, based on the weight of the phosphate.

20. The gel of claim 19, comprising between about 10% and about 20% by weight of pyrophosphate based on the weight of the phosphate.

21. The gel of any one of claims 1 to 20, further comprising on or more of corn oil, sodium metaphosphate, sodium pyrophosphate, sodium citrate, xantham gum, sodium alginate, a carboxylate salt, a carboxylate acid, or chitosan.

22. The gel of any one of claims 1 to 21, further having loaded therein a bioactive substance.

23. The gel of any one of claims 1 to 22, being non-toxic.

24. The gel of any one of claims 1 to 23, being thixotropic.

25. The gel of claim 24, having a liquefaction stress of about 50 Pa or less.

26. The gel of claim 25, having a liquefaction stress between about 30 and about 40 Pa.

27. The gel of any one of claims 24 to 26, having a recovery time of 10 seconds or less.

28. The gel of claim 27, having a recovery time of about 6 seconds.

29. The gel of any one of claims 1 to 28, being bioadhesive.

30. The gel of any one of claims 1 to 29, being bioresorbable.

31. A dehydrated or partially dehydrated magnesium phosphate gel comprising the gel of any one of claims 1 to 30, wherein at least part of the water in the dispersing phase is replaced by an organic liquid once the gel is formed.

32. The dehydrated or partially dehydrated gel of claim 31, wherein all of the water in the dispersing phase is replaced by the organic liquid.

33. The dehydrated or partially dehydrated gel of claim 31 or 32, wherein the organic liquid is ethanol or glycerol.

34. The gel of any one of claims 1 to 33 being dried so as to form a xerogel.

35. The gel of claim 34, being in the form of a membrane.

36. The gel of claim 35, wherein the membrane is translucent.

37. A method of manufacturing the gel of any one of claims 1 to 36, the method comprising:
(a) providing a first aqueous solution comprising sodium hydroxide,
(b) providing a second aqueous solution comprising phosphoric acid or monomagnesium phosphate,
(c) dissolving in the second solution, magnesium hydroxide or trimagnesium phosphate, and optionally calcium chloride or calcium hydroxide, thereby producing a third solution,
(d) mixing together the second and third solutions, thereby producing the gel, wherein the second and third solutions provide the gel with phosphate ions (PO₄³⁻), a divalent cation, and sodium ions (Na⁺) at mole fractions of about 0.25 to about 0.375, about 0.125 to about 0.5, and about 0.25 to about 0.5, respectively, wherein the divalent cation is magnesium (Mg²⁺) or a mixture of magnesium and calcium (Ca²⁺), the mixture comprising up to 30% by weight of calcium based on the total weight of the mixture.

38. The method of claim 37, wherein the mixing at step (d) occurs within 10 minutes of the dissolution at step (c).

39. The method of claim 37 or 38, wherein the second solution comprises phosphoric acid.

40. The method of any one of claims 37 to 39, wherein at step (c), magnesium hydroxide only is dissolved in the second solution.

41. The method of any one of claims 37 to 40, being carried out at room temperature.

42. The method of any one of claims 37 to 40, further comprising filtering and washing the gel.

43. The method of any one of claims 37 to 42, further comprising replacing at least part of the water in the dispersing phase by an organic liquid once the gel is formed.

44. The method of claim 43, wherein all of the water in the dispersing phase is replaced by the organic liquid.

45. The method of claim 43 or 44, wherein the organic liquid is ethanol or glycerol.

46. The method of any one of claims 37 to 45, further comprising loading a bioactive molecule in the gel.

47. The method of any one of claims 37 to 46, further comprising drying the gel to form a xerogel.

48. The method of claim 47, wherein the drying in carried out at room temperature.

49. Use of a gel according to any one of claims 1 to 36 as a bioactive substance delivery agent.

50. The use of claim 49, wherein the bioactive substance is an antibiotic.

51. The use of claim 49 or 50, wherein the delivery agent is for the treatment of a periodontal disease.

52. The use of claim 51, wherein the delivery agent is for the treatment of peri-implantitis.

53. The use of any one of claims 49 to 52, wherein the delivery agent is an adhesive delivery agent.

54. The use of claim 53, wherein the adhesive delivery agent is a mucosa adhesive delivery agent.

55. The use of claim 54, wherein the delivery agent is for transmucosal delivery of the bioactive substance.

56. The use of any one of claims 49 to 54, wherein the delivery agent is for topical delivery of the bioactive molecule.

57. The use of claim 49 or 50, wherein the delivery agent is an injectable delivery agent.

58. The use of claim 49 or 50, wherein the delivery agent is intended for oral administration.

59. The use of any one of claims 49 to 58, where the bioactive substance is a drug.

60. The use of claim 59, wherein the bioactive substance is an antiobiotic.

61. Use of a gel according to any one of claims 1 to 36 in wound care.

62. Use of a gel according to any one of claims 1 to 36 in a drug-eluting medical device.

63. Use of a gel according to any one of claims 1 to 36 in a coating.

64. Use of a gel according to any one of claims 1 to 36 as a coating.

65. A method of delivering a bioactive substance, the method comprising formulating the bioactive substance and a gel according to any one of claims 1 to 36 into a dosage form, and administering the dosage form.

66. The method of claim 65, wherein the dosage form is an adhesive dosage form.

67. The method of claim 66, wherein the adhesive dosage form is a mucosa adhesive dosage form.

68. The method of claim 67, wherein the dosage form delivers the bioactive substance via transmucosal absorption.

69. The method of any one of claims 65 to 67 wherein the dosage form delivers the bioactive substance via topical absorption.

70. The method of claim 65 wherein the dosage form is an injectable dosage form.

71. The method of claim 65, wherein the dosage form is an oral dosage form.

72. The method of any one of claims 65 to 71, where the bioactive substance is a drug.

73. A method of promoting healing of a wound, the method comprising applying a gel according to any one of claims 1 to 36 to the wound.

74. A drug-eluting medical device comprising a gel according to any one of claims 1 to 36.

75. A coating comprising a gel according to any one of claims 1 to 36.
